(19) United States
(12) Patent Application Publication
(10) Pub. No.: US 2008/0177379 A1
(43) Pub. Date: Jul. 24, 2008

(54) Single continuous piece prosthetic tubular aortic conduit and method for manufacturing the same

(76) Inventor: George Du, Fairfield, NJ (US)

Correspondence Address: J. Gary Mohr (Datascope), 14 Philips Parkway, Montvale, NJ, 07645, US

(21) Appl. No.: 11/655,438

(22) Filed: Jan. 19, 2007

(51) Int. Cl. A61F 2/06

(52) U.S. Cl. 623/1.51

(57) ABSTRACT

A prosthetic aortic conduit for replacing a root portion of an aorta is provided. The conduit comprises a continuous tubular conduit along a substantially common axis. A portion of the tubular conduit does not substantially deform in a longitudinal direction and has resilient means which allow said another portion of the conduit to be expandable in a lateral direction. The portion that is able to deform laterally mimics the function of the sinuses of Valsalva. The method of manufacturing such a conduit comprises the steps of having a continuous weave of rows of yarn or the equivalent with a change in tightness of the rows so that in some portion of the conduit it is expandable in the lateral direction and in some portion of the conduit it is expandable in the longitudinal direction.

### RELATED APPLICATIONS

This application incorporates by reference and claims priority to provisional application No. 60/760,017.

### BACKGROUND OF THE INVENTION

The normal internal human aortic root conduit is provided with a sinus portion which has three sinuses (bulges) which surround the aortic valve. These sinuses are called sinuses of Valsalva and are arranged so that the cross-section of the sinus portion has a generally trefoil shape. The diameter and orifice area of the root are greater at the level of the sinus, decrease slightly at the base, but significantly decrease (by 20%) at the level of the sinotubular junction (where the sinus portion connects to the ascending portion of the aorta which supports the two iliac arteries).

The sinotubular junction or sinus ridge and the sinuses of Valsalva are known to be crucial for the normal function of the aortic valve. The sinus ridge is important in causing initial fluid flow eddies inside the sinuses of Valsalva (see Bellhouse B J: Velocity and pressure distributions in the aortic valve. J Fluid Mech 1969; 37(3): 587-600 and Bellhouse B. J.: The fluid mechanics of the aortic valve. In: Ionescu M. L., Ross D. N., Woller G. H., eds. Biological tissue heart replacement. London: Butterworth-Heinemann, 1972:32-8). During systole, the aortic valve opens and the eddy currents created prevent the leaflets of the aortic valve from impacting on the aortic wall. Then, at the end of systole, the eddy currents inside the sinuses cause the leaflets of the aortic valve to become almost closed. Furthermore, the sinus curvature is very important in sharing stress with the leaflet. It has been demonstrated that during diastole the sinus walls move outwardly (increasing its circumferential curvature by 16%) taking up part of the load placed on the leaflet. Further it is known (see (Thubrikar M. J., Nolan S. P., Aouad J., Deck D.; Stress sharing between the sinus and leaflets of canine aortic valve. Ann Thorac Surg 1986; 42(4):434-40)) that the longitudinal length of the sinus changes very little or does not change at all during the cardiac cycle. In other words during the functioning of the aortic valve the sinus moves up and down as a whole without changing its length.

The standard surgical approach in patients with ascending aortic aneurysm or dissection involving the aortic root and associated with aortic valve disease is the replacement of the aortic valve and ascending aorta by means of a composite and valved graft onto which are reattached the two coronary arteries as originally described by Bentall and de Bono in their classical paper (Bentall H. H., De Bono A.: A technique for complete replacement of the ascending aorta, Thorax 1968; 23: 338-9). The “open” (Carrel button) method of coronary reimplantation was later recommended to decrease the tension on the coronary ostia while minimizing the risk of late false aneurysm formation. This “Carrel button” method has already reduced the incidence of pseudoaneurysm formation mainly through the reduction of the tension on the ostial anastomoses (see Svensson L. G.; Crawford E. S.; Hess K. R.; Coselli J. S.; Safi H. J.; Composite valve graft replacement of the proximal aorta: comparison of techniques in 348 patients. Ann Thorac Surg 1992, 54(3) 427-370). A modification of the standard technique was also introduced by Cabrol et al (Cabrol C, Pavie A, Gandjbakhch I. et al: Complete replacement of the ascending aorta with reimplantation of the coronary arteries. New Surgical approach, J Thorac Cardiovasc Surg 1981: 81; 309-15) for those cases of difficult presentation (low lying coronary ostia, calcified coronary ostia, tissue fibrosis in redo cases) where the coronary ostia are reattached to the aortic conduit by interposition of a small conduit.

If the aortic valve leaflets are normal, a valve-sparing aortic root remodeling procedure which keeps the natural patient valve on site is a reasonable alternative in certain individuals. David and Feindel (David T. E., Feindel C. M.: An aortic valve-sparing operation for patients with aortic incompetence and aneurysm of the ascending aorta, J Thorac Cardiovasc Surg 1992; 103(4): 617-21) described a surgical technique where the dilated aortic root is replaced with a tube made of DACRON fibers and the native aortic valve is integrated within the graft. This method is generally known as the “Tirone David Type I aortic valve sparing procedure”. However, the lack of sinuses in a straight tube graft was found to negatively influence proper valve function, with the consequent risk of decreasing valve longevity (Kunzelman K. S., Grande K. J., David T. E., Cochran R. P., Verrier E. D.: Aortic root and valve relationships. Impact on surgical repair J Thorac Cardiovascular Surg 1995; 109(2): 345-51).

Thus in the Tirone David Type I technique for valve sparing operations, the use of a straight tube without a sinus component raises several problems: opening and closing of the native valve is not optimal. For example, upon valve opening, the leaflets might impact on the graft and be potentially damaged. The absence or delay in eddy current formation might alter valve closure causing some regurgitation. Furthermore, the diastolic stress is borne only by the leaflet and is not shared with the sinuses causing a potential decrease in leaflet longevity.

An optimal design for root replacement should therefore incorporate sinuses and a sinotubular junction and further refinement of the technique consisted of trimming one end of the aortic tube graft to produce three separate extensions designed to replace the three sinuses. The reshaped DACRON tube was then sutured to the aortic valve remnants (see David T. E., Feindel C. M., Bos J.: Repair of the aortic valve in patients with aortic insufficiency and aortic root aneurysm. J Thorac Cardiovasc Surg 1995; 109(2):345-51) to obtain a final configuration resembling more closely the native aortic root. A similar technique was also described by Yacoub el al (Saram M. A., Yacoub M.: Remodeling of the aortic valve annulus. J Thorac Cardiovasc Surg 1993; 105(3): 435-8) several years previously.

In U.S. Pat. No. 5,139,515 it was proposed to provide an aortic graft having a lower portions provided with “bulges” apparently mimicking the sinuses of Valsalva. However no method to produce such a conduit for use in aortic surgery is described in the patent. U.S. Pat. No. 5,139,515 described a conduit having an “annular wall of a crimped material similar to that of conventional prosthesis”. No indication is actually given of how to obtain the “annually-spaced radially outward bulges” mimicking the sinuses. Moreover the drawings clearly show that the conduit, including the sinus portion, is provided along its whole length with corrugations which lie perpendicularly to the longitudinal axis of the prosethesis, and which impart longitudinal elasticity to the whole of the conduit. Upon implantation, the graft cannot expand radially outwardly, but has the potential to move and extend in the longitudinal direction of the longitudinal axis of the prosthesis.

Or as disclosed in U.S. Pat. No. 6,352,554, a conduit may comprise two distinct tubular portions having a common axis. The first upper portion is made form a standard aortic conduit and is provided with cicumfrerentially extending corrugations successively provided along the axis of the tubular first portion. The second lower portion, or skirt portion is a tube which can be made of the same material as the first portion (that is, any suitable biocompatible material, but preferably DACRON or PTFE) but which is provided with longitudinally extending pleats or corrugations. Each of these corrugations extends in the general direction of the longitudinal axis of the prosthesis and is positioned substantially perpendicularly to the circumferential corrugations of the first portion.

The proximal end of skirt portion 14 is attached to the distal end portion of the first portion 12 so the two connected portions have essentially the same lumen and form the tubular conduit 10.

Notwithstanding the above it is still preferred to have a single conduit, that can limit leakage, and avoid the need to connect two or more tubes to form the conduit.

Therefore there is still a need for an effective prosthetic conduit to replace the aortic root while providing all the advantages of the natural sinuses of Valsalva.

### SUMMARY OF THE INVENTION

It is therefore one of the objects of the invention to provide a prosthetic aortic conduit which overcomes the drawbacks mentioned above and which upon implantation has the ability to expand radially outwardly whilst maintaining a degree of flexibility in the longitudinal direction.

It is another object of the invention to provide a conduit which is specifically designed to closely mimic the sinuses of Valsalva.

A first object of the invention is a prosthetic aortic conduit for replacing a root portion of an aorta which comprises a first tubular portion and a second tubular portion connected together along a substantially common axis. The second tubular portion does not substantially deform in a longitudinal direction and has an expansion portion in a lateral direction.

It is preferred that the prosthetic aortic conduit be made of polyester or PTFE material or any other material capable of being woven.

It is further preferred that another portion of the conduit of the invention comprises annular corrugations successively provided along the longitudinal axis of said conduit.

It is further preferred that the first and second portions of the conduit be made of a single woven tube along a common axis.

It is further preferred that the conduit is provided with a third tubular portion which is part of the continuous conduit provided with resilient means which allows expansion of said third portion in a longitudinal direction.

Where a third portion of the conduit is required or used the weave will simply be continued form the second portion of the conduit.

### DESCRIPTION OF THE DRAWINGS

FIG. 1 is a representation of a known aortic conduit, showing corrugations which lie traverse to the longitudinal axis of the prosthesis;

FIG. 2 is a representation of another known aortic conduit:

FIG. 3 is a prosthetic aortic conduit according to the first preferred embodiment of the invention;

FIG. 4 is a prosthetic aortic conduit according to a second preferred embodiment of the invention; and

FIG. 5 a side view of the weave showing a portion where the individual rows of weave are close and a second portion where the individual rows of weave are further apart.

### DESCRIPTION OF PREFERRED EMBODIMENTS OF THE INVENTION

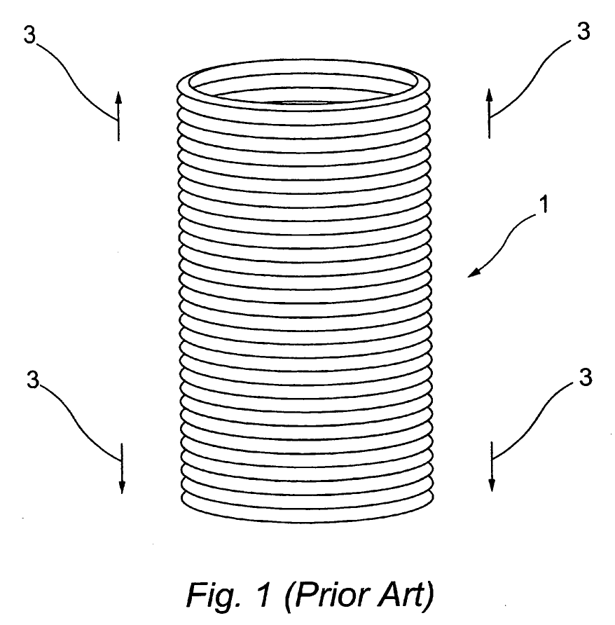

FIG. 1 shows a standard aortic conduit 1 of the type currently used in aortic surgery. This conduit is made of DACRON but any suitable biocompatible material such as polytetrafluoroethylene (PTFE) could be used. This standard aortic conduit 1 includes circumferentially extending pleats so that the corrugations lie perpendicular to the longitudinal axis of the prosthesis. These corrugations provide a degree of expansion in the longitudinal direction (indicated by the black arrows 3 in FIG. 1) and the conduit 1 can therefore significantly increase its length.

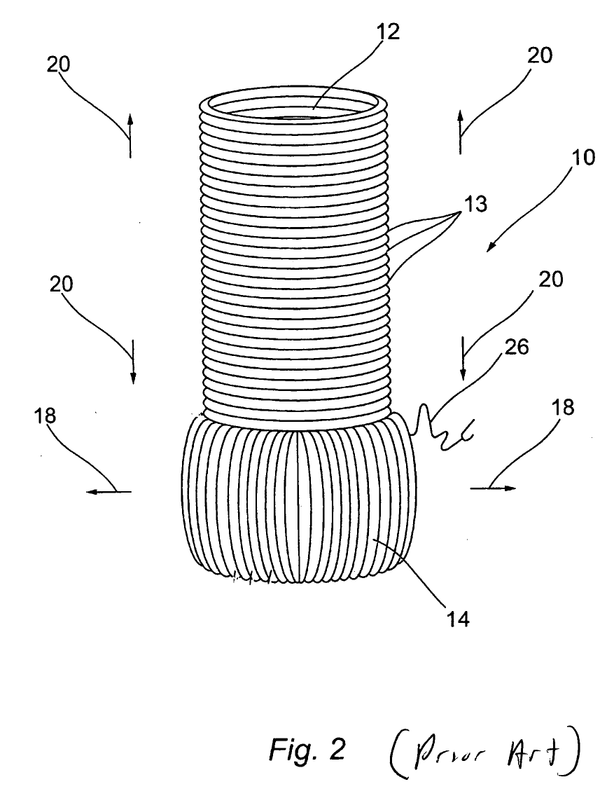

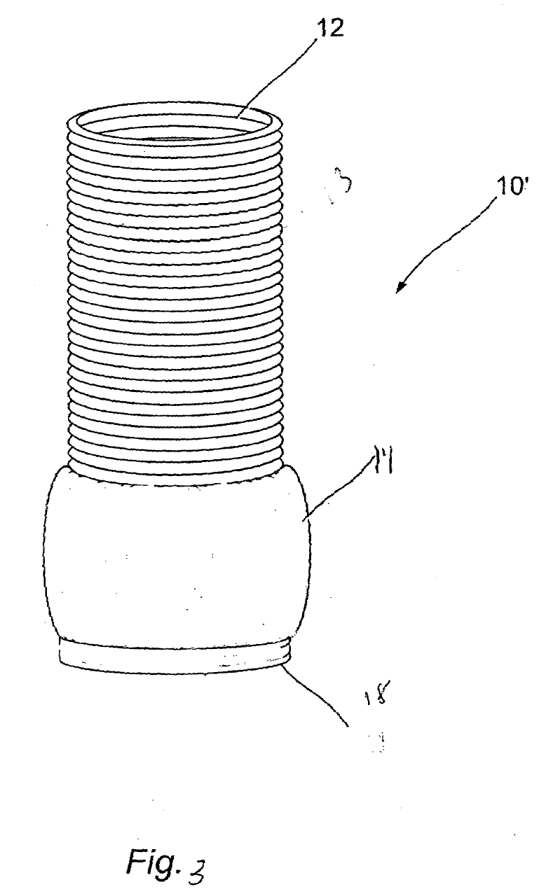

FIG. 2 shows a preferred embodiment of the conduit of the invention. The conduit 10 comprises two distinct tubular portions having a common axis. The first upper portion 12 is made from a standard aortic conduit similar to the one shown in FIG. 1 and is provided with circumferentially extending corrugations 13 successively provided along the axis of the tubular first portion 12. The second lower portion, or skirt portion, 14 is a tube which can be made of the same material as the first portion (that is, any suitable biocompatible material) but which has longitudinally extending pleats or corrugations 16.

The proximal end of skirt portion 14 is attached to the distal end portion of the first portion 12 so the two connected portions have essentially the same lumen and form the tubular conduit 10.

As shown in FIG. 3, which is one embodiment of the preferred invention, the first portion 12 and the skirt portion 14 with their respective corrugations 13 orientated at an angle of about 90 degrees will act, upon implantation, as a “sinotubular junction” witch it internal diameter will be significantly less than the internal diameter of its lower part, namely second portion 14. Once the prosthetic aortic conduit 10 is in place the internal diameter of the skirt portion 14 will vary during the cardiac cycle (systole/diastole) as in the natural aortic root. Thus, the skirt portion 14, when filled with blood under pressure, will stretch in the direction traverse to the longitudinal axis of the prostheses (the lateral direction) mimicking the “sinuses of Valsalva”. However, the skirt portion 14 does not, however, allow that section of the prosthesis to increase in length and has a collar 18 for attachment purposes.

Thus the skirt portion 14 can move and expand in a lateral direction only, while the first portion 12 of conduit 10 can extend in the longitudinal direction only. The resiliency of the skirt portion 14 in the general lateral direction is shown in FIG. 2 by the arrows 18 and the expansion of the first portion 12 in the general longitudinal direction is shown by the arrows 2.

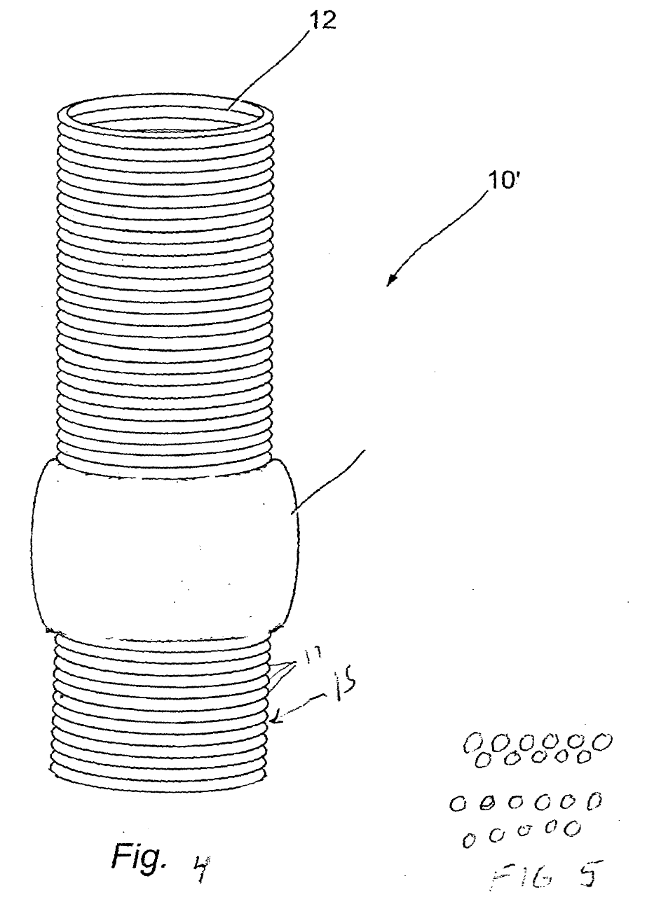

In an alternative embodiment shown I FIG. 4, a third tubular portion 15 is attached to the distal end of the skirt portion 14. The third tubular portion 15 is aligned on the same common axis as the first and second portions 12 and 14. The third portion 15 is advantageously made of any length that is desired. It is typically made of DACRON or similar material or any material that may be weaned and is provided with circumferentially extending corrugations or pleats 17 in the same manner as the first portion 20.

### Preferred Method of Manufacture of a Conduit According to the Invention

The conduit 10 may be either manufactured as shown in FIG. 5, by changing the tightness of each row of the individual weave material 30 to create the desired diameter of the conduit 10.

The device and method of manufacture according to the present invention have been described in the foregoing specification. However, the invention which is intended to be protected is not to be construed as limited to the particular embodiments disclosed. Further, the embodiments described herein are to be regarded as illustrative rather that restrictive. Variations, changes and equivalents may be made by others without departing from the spirit of the present invention. Accordingly, it is expressly intended that all such variation, changes and equivalents which fall within the spirit and scope of the present invention as defined in the claims, be embraced thereby.

I claim:

1. A prosthetic aortic conduit for replacing a root portion of an aorta, said conduit having first and second ends, wherein said conduit comprises a continuous weave wherein the tightness of the weave determines what portion of the conduit will only expand in the longitudinal direction and what portion of the conduit will expand in the lateral direction.

2. The prosthetic aortic conduit of claim 1, wherein said conduit is made of DACRON or PTFE material.

4. The prosthetic aortic conduit of claim 1, wherein said first portion resilient means comprises annular corrugations successively provided along the longitudinal axis of said first portion.

5. The prosthetic aortic conduit of claim 1, wherein a third tubular portion is connected to the second portion along said substantially common axis and wherein said third tubular portion is provided with resilient means which allow expansion of said third portion in a longitudinal direction.

6. A method of manufacturing a prosthetic aortic conduit having first and second ends, which comprises the following steps:
a) providing a first tubular woven conduit suitable for use in heart surgery, said first conduit having a longitudinal axis and first resilient means allowing some expansion in the longitudinal direction only; and
b) continuing the weave of the first tubular conduit to form a second tubular sinus conduit suitable for use in heart surgery, said second sinus conduit having a circumference and second resilient means which allows some expansion in the lateral direction only; so that the first tubular portion forms the first end of the prosthetic aortic conduit.
